(19) United States
(12) Patent Application Publication
(10) Pub. No.: US 2011/0112155 A1
(43) Pub. Date: May 12, 2011

(54) METHODS FOR TREATING CANCER IN PATIENTS HAVING BREAST CANCER RESISTANCE PROTEIN OVEREXPRESSION

(75) Inventors: Francis Y Lee, Yardley, PA (US); Jinping Gan, Princeton, NJ (US); Hong Shen, Belle Mead, NJ (US)

(73) Assignee: Bristol-Myers Squibb Company

(21) Appl. No.: 12/812,777

(22) PCT Filed: Jan. 15, 2009
(86) PCT No.: PCT/US2009/031052

§ 371 Date: Jul. 14, 2010

Related U.S. Application Data
(60) Provisional application No. 61/021,149, filed on Jan. 15, 2008.

(51) Int. Cl. A61K 31/427; A61P 35/00; C12Q 1/68; C12Q 1/02; G01N 33/53

(52) U.S. Cl. 514/365; 435/6; 435/29; 435/7.21

(57) ABSTRACT

A method for treating cancer comprising identifying a mammal that overexpresses breast cancer resistance protein; and administering to said mammal a pharmaceutical composition comprising a therapeutically effective amount of ixabepilone. In one aspect, the mammal is not administered an agent that is susceptible to breast cancer resistance protein overexpression resistance. In another aspect, the cancer is breast and/or lung cancer.

This application claims benefit to provisional application U.S. Ser. No. 61/021,149, filed Jan. 15, 2008; under 35 U.S.C. 119(e). The entire teachings of the referenced applications are incorporated herein by reference.

### FIELD OF THE INVENTION

The present invention relates generally to the field of pharmacogenomics, and more specifically to methods and procedures to determine drug sensitivity in patients to allow the identification of individualized genetic profiles which will aid in treating diseases and disorders.

### BACKGROUND OF THE INVENTION

The 72-kDa breast cancer resistance protein (BCRP) is the second member of the subfamily G of the human ATP binding cassette (ABC) transporter superfamily and thus also designated as ABCG2. Unlike P-glycoprotein and MRP1, which are arranged in 2 repeated halves, BCRP is a half-transporter consisting of only 1 nucleotide binding domain followed by 1 membrane-spanning domain. Current experimental evidence suggests that BCRP may function as a homodimer or homotetramer. Overexpression of BCRP is associated with high levels of resistance to a variety of anticancer agents, including anthracyclines, mitoxantrone, and the camptothecins, by enhancing drug efflux. BCRP expression has been detected in a large number of hematological malignancies and solid tumors, indicating that this transporter may play an important role in clinical drug resistance of cancers. In addition to its role to confer resistance against chemotherapeutic agents, BCRP actively transports structurally diverse organic molecules, conjugated or unconjugated, such as estrone-3-sulfate, 17β-estradiol 17-(β-D-glucuronide), and methotrexate. BCRP is highly expressed in the placental syncytiotrophoblasts, in the apical membrane of the epithelium in the small intestine, in the liver canalicular membrane, and at the luminal surface of the endothelial cells of human brain microvessels. This strategic and substantial tissue localization indicates that BCRP also plays an important role in absorption, distribution, and elimination of drugs that are BCRP substrates. See, Mao et al., AAPS Journal, 07(01):E118-E133 (2005).

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (Cockett, M. et al., Current Opinion in Biotechnology, 11:602-609 (2000)).

The ability to determine which patients are responding to anti-angiogenesis therapies (such as microtubule-stabilizing agents) or predict drug sensitivity in patients is particularly challenging because drug responses reflect not only properties intrinsic to the target cells, but also a host's metabolic properties. Efforts to use genetic information to predict or monitor drug response have primarily focused on individual genes that have broad effects, such as the multidrug resistance genes mdr1 and mrp1 (Sonneveld, P., J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (Khan, J. et al., Cancer Res., 58:5009-5013 (1998); Alizadeh, A. A. et al., Nature, 403:503-511 (2000); Bittner, M. et al., Nature, 406:536-540 (2000); Khan, J. et al., Nature Medicine, 7(6):673-679 (2001); and Golub, T. R. et al., Science, 286:531-537 (1999); Alon, U. et al., Proc. Natl. Acad Sci. USA, 96:6745-6750 (1999)). Such technologies and molecular tools have made it possible to monitor the expression level of a large number of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588 to Ashby et al.).

Recent studies demonstrate that gene expression information generated by microarray analysis of human tumors can predict clinical outcome (van't Veer, L. J. et al., Nature, 415:530-536 (2002); Shipp, M. et al., Nature Medicine, 8(1):68-74 (2002); Glinsky, G. et al., J. Clin. Invest., I13(6):913-923 (2004)). These findings bring hope that cancer treatment will be vastly improved by better predicting and monitoring the response of individual tumors to therapy.

Microtubule-stabilizing agents, such as ixabepilone (IXEMPRA™) and paclitaxel (TAXOL®), are commonly used for the treatment of many types of cancer, including breast and lung cancer.

Needed are new and alternative methods and procedures to determine drug sensitivity or monitor response in patients to allow the development of individualized diagnostics which are necessary to treat diseases and disorders based on patient response at a molecular level, particularly for breast and lung cancer patients.

### SUMMARY OF THE INVENTION

The invention provides methods and procedures for determining patient sensitivity to one or more microtubule-stabilizing agents.

In one aspect, the invention relates to a method for treating cancer comprising identifying a mammal that overexpresses breast cancer resistance protein; and administering to said mammal a pharmaceutical composition comprising a therapeutically effective amount of ixabepilone, either alone or in combination with another agent. In one aspect, the mammal is not administered an agent that is susceptible to breast cancer resistance protein overexpression resistance. In another aspect, the cancer is breast and/or lung cancer. In yet another aspect, the mammal further overexpresses at least one of BRCP (ABCG2), beta-tubulin III (TUBB3), MDR1, MRP1, and a beta-tubulin mutant. In one aspect, the mammal is a human.

The present invention provides a method of screening a biological sample, for example cells that do not respond, or that have stopped responding, or that have a diminished response, to one or more microtubule-stabilizing agents. For example, the present invention provides a method of screening cells from an individual suffering from cancer who is either being treated with one or more microtubule-stabilizing agents or is naïve to said agents, and whose cells do not respond or have stopped responding or that have a diminished response to one or more microtubule-stabilizing agents, for overexpression of breast cancer resistance protein relative to a standard. If breast cancer resistance protein overexpression is present, administration of a therapeutically acceptable amount of ixabepilone, alone or in combination with one or more microtubule-stabilizing agents and/or other agent, such as a CTLA4 antagonist, is warranted to inhibit proliferation of said cells. Wherein said cancer is breast and/or lung cancer.

The present invention provides a method of screening a biological sample, for example cells that do not respond, or that have stopped responding, or that have a diminished response, to one or more microtubule-stabilizing agents. For example, the present invention provides a method of screening cells from an individual suffering from cancer who is either being treated with one or more microtubule-stabilizing agents or is nave to said agents, and whose cells do not respond or have stopped responding or that have a diminished response to one or more microtubule-stabilizing agents, for overexpression of breast cancer resistance protein and beta-tubulin III relative to a standard. If breast cancer resistance protein and beta-tubulin III overexpression is present, administration of a therapeutically acceptable amount of ixabepilone, alone or in combination with one or more microtubule-stabilizing agents and/or other agent, such as a CTLA4 antagonist, is warranted to inhibit proliferation of said cells. Wherein said cancer is breast and/or lung cancer.

The present invention provides a method of screening a biological sample, for example cells that do not respond, or that have stopped responding, or that have a diminished response, to one or more microtubule-stabilizing agents. For example, the present invention provides a method of screening cells from an individual suffering from cancer who is either being treated with one or more microtubule-stabilizing agents or is naïve to said agents, and whose cells do not respond or have stopped responding or that have a diminished response to one or more microtubule-stabilizing agents, for overexpression of breast cancer resistance protein and MDR1 relative to a standard. If breast cancer resistance protein and MDR1 overexpression is present, administration of a therapeutically acceptable amount of ixabepilone, alone or in combination with one or more microtubule-stabilizing agents and/or other agent, such as a CTLA4 antagonist, is warranted to inhibit proliferation of said cells. Wherein said cancer is breast and/or lung cancer.

The present invention provides a method of screening a biological sample, for example cells that do not respond, or that have stopped responding, or that have a diminished response, to one or more microtubule-stabilizing agents. For example, the present invention provides a method of screening cells from an individual suffering from cancer who is either being treated with one or more microtubule-stabilizing agents or is naïve to said agents, and whose cells do not respond or have stopped responding or that have a diminished response to one or more microtubule-stabilizing agents, for overexpression of breast cancer resistance protein and MRP1 relative to a standard. If breast cancer resistance protein and MRP1 overexpression is present, administration of a therapeutically acceptable amount of ixabepilone, alone or in combination with one or more microtubule-stabilizing agents and/or other agent, such as a CTLA4 antagonist, is warranted to inhibit proliferation of said cells. Wherein said cancer is breast and/or lung cancer.

The present invention provides a method of screening a biological sample, for example cells that do not respond, or that have stopped responding, or that have a diminished response, to one or more microtubule-stabilizing agents. For example, the present invention provides a method of screening cells from an individual suffering from cancer who is either being treated with one or more microtubule-stabilizing agents or is naïve to said agents, and whose cells do not respond or have stopped responding or that have a diminished response to one or more microtubule-stabilizing agents, for overexpression of breast cancer resistance protein and beta-tubulin mutant relative to a standard. If breast cancer resistance protein and beta-tubulin mutant overexpression is present, administration of a therapeutically acceptable amount of ixabepilone, alone or in combination with one or more microtubule-stabilizing agents and/or other agent, such as a CTLA4 antagonist, is warranted to inhibit proliferation of said cells. Wherein said cancer is breast and/or lung cancer.

The diagnostic methods of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the biomarker(s).

The invention also provides an isolated TUBB3 biomarker, an isolated BRCP (ABCG2) biomarker, an isolated MDR1 (ABCB1) biomarker, an isolated MRP1 (ABCC1) biomarker, and tubulin mutation biomarkers. The biomarkers of the invention include nucleotide and amino acid sequences of full-length TUBB3, BRCP (ABCG2), MDR1 (ABCB1), MRP1 (ABCC1), and beta-tubulin mutations, as well as fragments and variants thereof.

The invention also provides a biomarker set comprising two or more biomarkers of the invention.

The invention also provides kits for measuring overexpression of breast cancer resistance protein, and uses thereof. The invention also provides antibodies, including polyclonal or monoclonal, directed breast cancer resistance protein, and uses thereof.

The invention will be better understood upon a reading of the detailed description of the invention when considered in connection with the accompanying figures.

### BRIEF DESCRIPTION OF THE DRAWINGS

FIG. 1 shows a schematic of the structure and mechanism of ixabepilone.

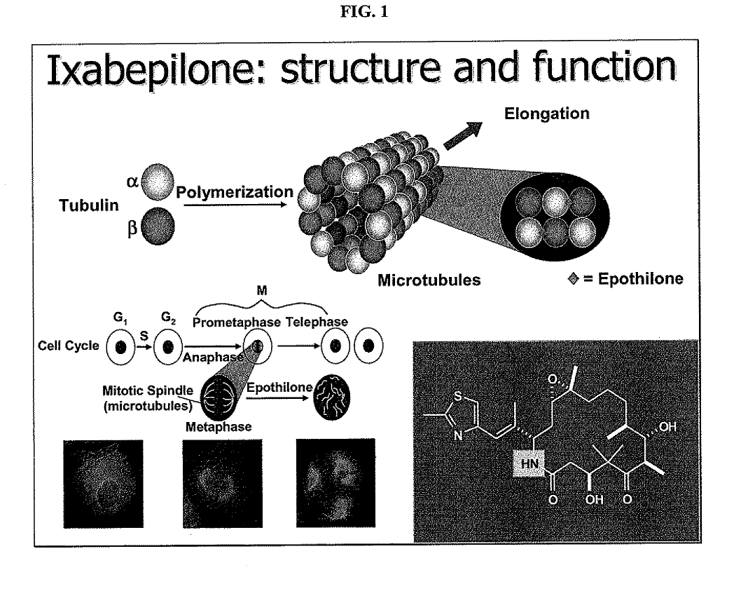

FIG. 2 illustrates the issue of drug resistance for taxanes as well as providing examples of several mechanisms of how such resistance may arise.

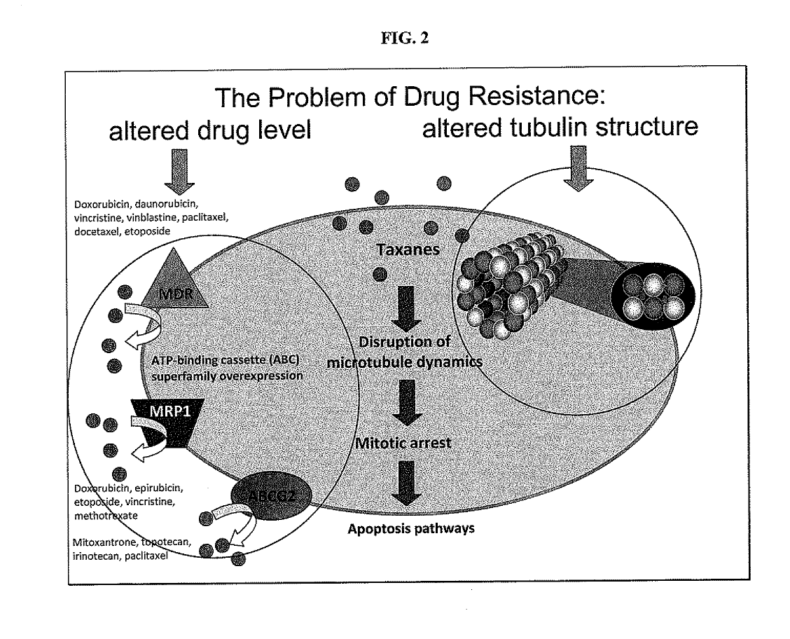

FIG. 3 shows the ability of ixabepilone to overcome multiple mechanisms of taxane-resistance.

FIG. 4 provides an illustration of ABC drug resistance transporters.

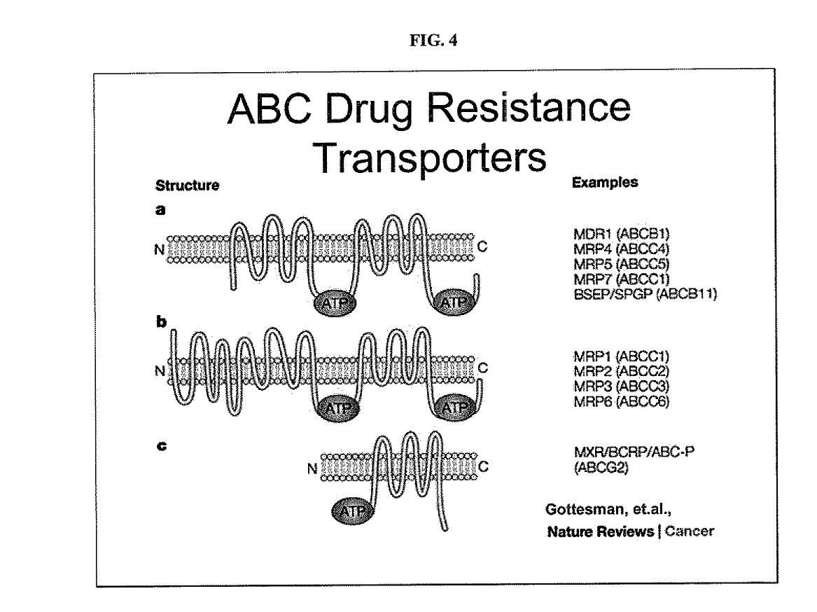

FIG. 5 shows the ability of ixabepilone to effectively overcome the BCRP overexpression-induced resistance to microtubulin stabilizers. BCRP overexpressing HEK293 cells are denoted as “BCRP/HEK 293 Cells”, while control HEK293 cells are denoted as “HEK 293 Cells”.

FIG. 6 shows the IC50 of paclitaxel, mitotropine, and ixabepilone in BCRP overexpressing HEK293 cells. As shown, ixabepilone had a very low IC50.

FIG. 7 shows the detection of BCRP overexpressing cells using Hoescht 33342 dye and flow cytometry, and the abolition of BCRP overexpressing cells subsequent to the administration of the BCRP inhibitor, Fumitremorgin C.

FIG. 8 shows the increased ability of ixabepilone to overcome BCRP-overexpression dependent resistance to taxanes in a human lung carcinoma cell line, H441.

### DETAILED DESCRIPTION OF THE INVENTION

As is known in the art, ixabepilone refers to a compound having the following structure (I):

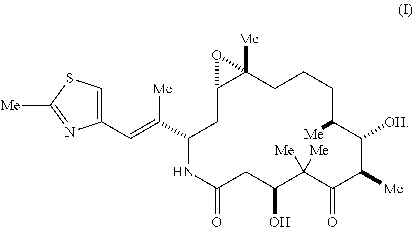

Compound (I) can also be referred to as (1S,3 S,7S,10R,11S,12S,16R)-7,11-dihydroxy-8,8,10,12,16-pentamethyl-3-[(1E)-1-methyl-2-(2-methyl-4-thiazolyl)pethenyl]-17-oxa-4-azabicyclo[14.1.0]heptadecane-5,9-dione in accordance with IUPAC nomenclature. Use of the term “(1S,3S,7S,10R,11S,12S,16R)-7,11-dihydroxy-8,8,10,12,16-pentamethyl-3-[(1E)-1-methyl-2-(2-methyl-4-thiazolyl)pethenyl]-17-oxa-4-azabicyclo[14.1.0]heptadecane-5,9-dione” encompasses (unless otherwise indicated) solvates (including hydrates) and polymorphic forms of the compound (I) or its salts, such as the forms of (I) described in U.S. Pat. No. 6,605,599, issued Aug. 12, 2003, incorporated herein by reference in its entirety and for all purposes. Pharmaceutical compositions of (1 S,3 S,7S, 10R,11 S,128,16R)-7,11-dihydroxy-8,8,10,12,16-pentamethyl-3-[(1E)-1-methyl-2-(2-methyl-4-thiazolyl)pethenyl]-17-oxa-4-azabicyclo[14.1.0]heptadecane-5,9-dione include all pharmaceutically acceptable compositions comprising (1S,3S,7S,10R,11S,12S,16R)-7,11-dihydroxy-8,8,10,12,16-pentamethyl-3-[(1E)-1-methyl-2-(2-methyl-4-thiazolyl)pethenyl]-17-oxa-4-azabicyclo[14.1.0]heptadecane-5,9-dione and one or more diluents, vehicles and/or excipients One example of a pharmaceutical composition comprising (1S,3S,7S,10R,11S,12S,16R)-7,11-dihydroxy-8,8,10,12,16-pentamethyl-3-[(1E)-1-methyl-2-(2-methyl-4-thiazolyl)pethenyl]-17-oxa-4-azabicyclo[14.1.0]heptadecane-5,9-dione is IXEMPRA™ (Bristol-Myers Squibb Company). IXEMPRA™ comprises (1S,3S,7S,10R,11S,12S,16R)-7,11-dihydroxy-8,8,10,12,16-pentamethyl-3-[(1E)-1-methyl-2-(2-methyl-4-thiazolyl)ethenyl]-17-oxa-4-azabicyclo[14.1.0]heptadecane-5,9-dione as the active ingredient, also referred to as ixabepilone, for IV infusion including inactive ingredients in the form of a diluent consisting of a sterile, non-pyrogenic of 52.8% (w/v) purified polyoxyethylated castor oil and 39.8% (w/v) dehydrated alcohol, USP.

Non-limiting examples of other epothilones for use in the methods and compositions of the present invention are encompassed by formula II:

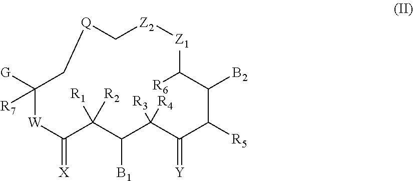

wherein:

Q is selected from the group consisting of:

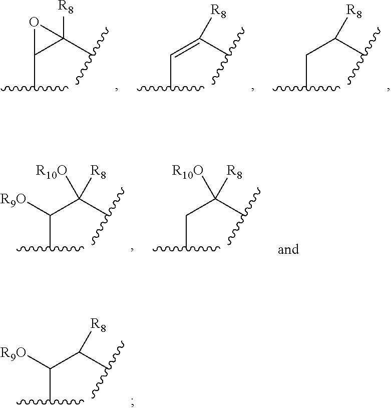

G is selected from the group consisting of alkyl, substituted alkyl, aryl, substituted aryl, heterocyclo,

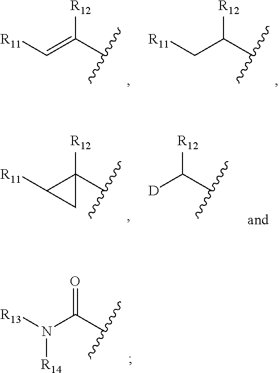

W is O or N R₁₅;

X is O or H, H;

Y is selected from the group consisting of O; H, OR₁₆; OR₁₇, OR₁₇; NOR₁₈; H, NHOR₁₉; H, NR₂₀R₂₁; H, H; and CHR₂₂; wherein OR₁₇, OR₁₇ can be a cyclic ketal;

Z₁ and Z₂ are independently selected from the group consisting of CH₂, O, NR₂₃, S, and SO₂, wherein only one of Z₁ and Z₂ can be a heteroatom;

B₁ and B₂ are independently selected from the group consisting of OR₂₄, OCOR₂₅, and O—C(═O)—NR₂₆R₂₇, and when B₁ is H and Y is OH, H, they can form a six-membered ring ketal or acetal;

D is selected from the group consisting of NR₂₈R₂₉, NR₃₀COR₃₁ and saturated heterocycle;

R₁, R₂, R₃, R₄, R₅, R₆, R₇, R₁₃, R₁₄, R₁₈, R₁₉, R₂₀, R₂₁, R₂₂, R₂₆ and R₂₇ are independently selected from the group consisting of H, alkyl, substituted alkyl, and aryl, and when R₁ and R₂ are alkyl can be joined to form a cycloalkyl, and when R₃ and R₄ are alkyl can be joined to form a cycloalkyl;

R₉, R₁₀, R₁₆, R₁₇, R₂₄, R₂₅ and R₃₁ are independently selected from the group consisting of H, alkyl, and substituted alkyl;

R₈, R₁₁, R₁₂, R₂₈, R₃₀, R₃₂, and R₃₃ are independently selected from the group consisting of H, alkyl, substituted alkyl, aryl, substituted aryl, cycloalkyl and heterocyclo; and

R₁₅, R₂₃ and R₂₉ are independently selected from the group consisting of H, alkyl, substituted alkyl, aryl, substituted aryl, cycloalkyl, heterocyclo, R₃₂C═O, R₃₃SO₂, hydroxy, O-alkyl or O-substituted alkyl; and pharmaceutically acceptable salts thereof and any hydrates, solvates or geometric, optical and stereo isomers thereof.

Formula III provides another example of an epothilone suitable for use in the methods and compositions of the present invention:

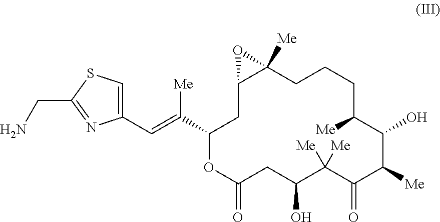

wherein:

P-Q is a C, C double bond or an epoxide;

G is

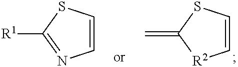

R is selected from the group of H, alkyl, and substituted alkyl;

R¹ is selected from the group consisting of:

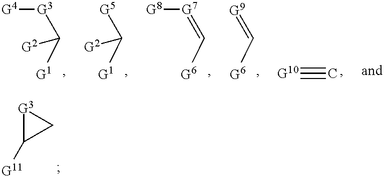

R² is

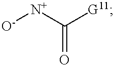

G¹ is selected from the group of H, halogen, CN, alkyl and substituted alkyl;

G² is selected from the group of H, alkyl, and substituted alkyl;

G³ is selected from the group of O, S, and NZ¹;

G⁴ is selected from the group of H, alkyl, substituted alkyl, OZ², NZ²Z³, Z²C═O, Z⁴SO₂, and optionally substituted glycosyl;

G⁵ is selected from the group of halogen, N₃, NCS, SH, CN, NC, N(Z¹)₃⁺ and heteroaryl;

G⁶ is selected from the group of H, alkyl, substituted alkyl, CF₃, OZ⁵, SZ⁵, and NZ⁵Z⁶;

G⁷ is CZ⁷ or N;

G⁸ is selected from the group of H, halogen, alkyl, substituted alkyl, OZ¹⁰, SZ¹⁰, NZ¹⁰Z¹¹;

G⁹ is selected from the group of O, S, —NH—NH— and —N═N—;

G¹⁰ is N or CZ¹²;

G¹¹ is selected from the group of H₂N, substituted H₂N, alkyl, substituted alkyl, aryl, and substituted aryl;

Z¹, Z⁶, Z⁹, and Z¹¹ are independently selected from the group H, alkyl, substituted alkyl, acyl, and substituted acyl;

Z² is selected from the group of H, alkyl, substituted alkyl, aryl, substituted aryl, and heterocycle;

Z³, Z⁵, Z⁸, and Z¹⁰ are independently selected from the group H, alkyl, substituted alkyl, acyl, substituted acyl, aryl, and substituted aryl;

Z⁴ is selected from the group of alkyl, substituted alkyl, aryl, substituted aryl, and heterocycle;

Z⁷ is selected from the group of H, halogen, alkyl, substituted alkyl, aryl, substituted aryl, OZ⁸, SZ⁸, and NZ⁸Z⁹; and

Z¹² is selected from the group of H, halogen, alkyl, substituted alkyl, aryl, and substituted aryl;

with the proviso that when R¹ is

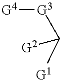

G¹, G², G³ and G⁴ cannot simultaneously have the following meanings:

G¹and G²=H, G³=O and G⁴=H or Z²C═O where Z²=alkyl group.

A preferred compound of Formula III of the invention is Formula

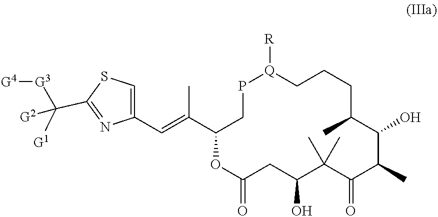

wherein the symbols have the following meaning:

P-Q is a C,C double bond or an epoxide;

R is a H atom or a methyl group;

G¹ is an H atom, an alkyl group, a substituted alkyl group or a halogen atom;

G² is an H atom, an alkyl group or a substituted alkyl group;

G³ is an O atom, an S atom or an NZ¹ group with Z¹ being an H atom, an alkyl group, a substituted alkyl group, an acyl group, or a substituted acyl group;

G⁴ is an H atom, an alkyl group, a substituted alkyl group, an OZ² group, an NZ²Z³ group, a Z²C═O group, a Z⁴SO₂ group or an optionally substituted glycosyl group with Z² being a H atom, an alkyl group, a substituted alkyl group, an aryl group, a substituted aryl group or a heterocyclic group;

Z³ an H atom, an alkyl group, a substituted alkyl group, an acyl group or a substituted acyl group; and

Z⁴ an alkyl, a substituted alkyl, an aryl, a substituted aryl or a heterocyclic group, with the proviso that G¹, G², G³ and G⁴ cannot have simultaneously the following meanings: G¹ and G²=H atom, G³=O atom and G⁴=H atom or Z²C═O with Z²=alkyl group.

A particularly preferred compound of Formula III is [1S-[1R*,3R* (E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4,17-dioxabicyclo[14.1.0]heptadecane-5,9-dione (Compound 4) and pharmaceutically acceptable salts thereof.

The phrase “microtubulin modulating agent” is meant to refer to agents that either stabilize microtubulin or destabilize microtubulin synthesis and/or polymerization.

Microtubulin modulatory agents either agonize or inhibit a cells ability to maintain proper microtubulin assemblies. In the case of paclitaxel (marketed as TAXOL®) causes mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)).

Ixabepilone is a semi-synthetic lactam analogue of patupilone that binds to tubulin and promotes tubulin polymerisation and microtubule stabilisation, thereby arresting cells in the G2/M phase of the cell cycle and inducing tumour cell apoptosis.

Thus, in a preferred embodiment, the therapeutic method of the invention comprises the administration of Formulas I, II, III, and/or IIIa or analogs thereof.

A preferred epothilone analog for use in the methods of the invention is a compound of Formula II:

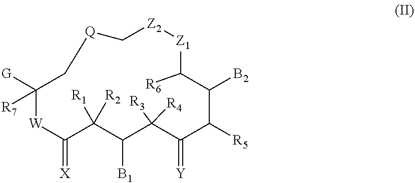

wherein:

Q is selected from the group consisting of:

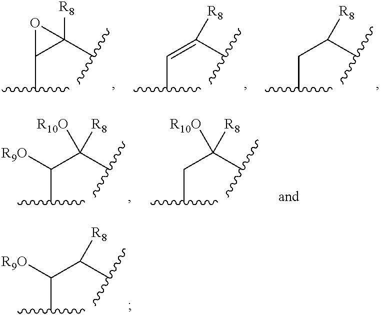

G is selected from the group consisting of alkyl, substituted alkyl, aryl, substituted aryl, heterocyclo,

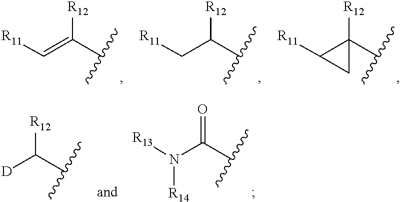

W is O or N R₁₅;

X is O or H, H;

Y is selected from the group consisting of O; H, OR₁₆; OR₁₇, OR₁₇; NOR₁₈; H, NHOR₁₉; H, NR₂₀R₂₁; H, H; and CHR₂₂; wherein OR₁₇, OR₁₇ can be a cyclic ketal;

Z₁ and Z₂ are independently selected from the group consisting of CH₂, O, NR₂₃, S, and SO₂, wherein only one of Z₁ and Z₂ can be a heteroatom;

B₁ and B₂ are independently selected from the group consisting of OR₂₄, OCOR₂₅, and O—C(═O)—NR₂₆R₂₇, and when B₁ is H and Y is OH, H, they can form a six-membered ring ketal or acetal;

D is selected from the group consisting of NR₂₈R₂₉, NR₃₀COR₃₁ and saturated heterocycle;

R₁, R₂, R₃, R₄, R₅, R₆, R₇, R₁₃, R₁₄, R₁₈, R₁₉, R₂₀, R₂₁, R₂₂, R₂₆ and R₂₇ are independently selected from the group consisting of H, alkyl, substituted alkyl, and aryl, and when R₁ and R₂ are alkyl can be joined to form a cycloalkyl, and when R₃ and R₄ are alkyl can be joined to form a cycloalkyl;

R₉, R₁₀, R₁₆, R₁₇, R₂₄, R₂₅ and R₃₁ are independently selected from the group consisting of H, alkyl, and substituted alkyl;

R₈, R₁₁, R₁₂, R₂₈, R₃₀, R₃₂, and R₃₃ are independently selected from the group consisting of H, alkyl, substituted alkyl, aryl, substituted aryl, cycloalkyl and heterocyclo;

R₁₅, R₂₃ and R₂₉ are independently selected from the group consisting of H, alkyl, substituted alkyl, aryl, substituted aryl, cycloalkyl, heterocyclo, R₃₂C═O, R₃₃SO₂, hydroxy, O-alkyl or O-substituted alkyl; and

pharmaceutically acceptable salts thereof and any hydrates, solvates or geometric, optical and stereoisomers thereof.

Another preferred epothilone for use in the present invention is a compound of Formula III:

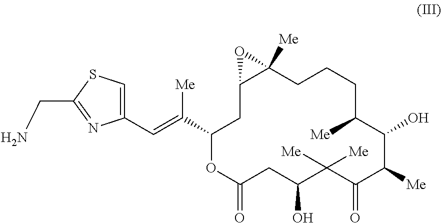

wherein:

P-Q is a C, C double bond or an epoxide;

G is

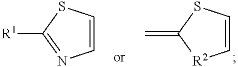

R is selected from the group of H, alkyl, and substituted alkyl;

R¹ is selected from the group consisting of:

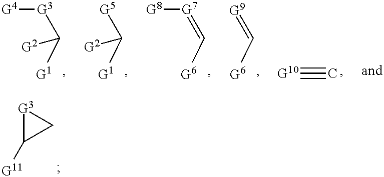

R² is

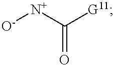

G¹ is selected from the group of H, halogen, CN, alkyl and substituted alkyl;

G² is selected from the group of H, alkyl, and substituted alkyl;

G³ is selected from the group of O, S, and NZ¹;

G⁴ is selected from the group of H, alkyl, substituted alkyl, OZ², NZ²Z³, Z²C═O, Z⁴SO₂, and optionally substituted glycosyl;

G⁵ is selected from the group of halogen, N₃, NCS, SH, CN, NC, N(Z¹)₃⁺ and heteroaryl;

G⁶ is selected from the group of H, alkyl, substituted alkyl, CF₃, OZ⁵, SZ⁵, and NZ⁵Z⁶;

G⁷ is CZ⁷ or N;

G⁸ is selected from the group of H, halogen, alkyl, substituted alkyl, OZ¹⁰, SZ¹⁰, NZ¹⁰Z¹¹;

G⁹ is selected from the group of O, S, —NH—NH— and —N═N—;

G¹⁰ is N or CZ¹²;

G¹¹ is selected from the group of H₂N, substituted H₂N, alkyl, substituted alkyl, aryl, and substituted aryl;

Z¹, Z⁶, Z⁹, and Z¹¹ are independently selected from the group H, alkyl, substituted alkyl, acyl, and substituted acyl;

Z² is selected from the group of H, alkyl, substituted alkyl, aryl, substituted aryl, and heterocycle;

Z³, Z⁵, Z⁸, and Z¹⁰ are independently selected from the group H, alkyl, substituted alkyl, acyl, substituted acyl, aryl, and substituted aryl;

Z⁴ is selected from the group of alkyl, substituted alkyl, aryl, substituted aryl, and heterocycle;

Z⁷ is selected from the group of H, halogen, alkyl, substituted alkyl, aryl, substituted aryl, OZ⁸, SZ⁸, and NZ⁸Z⁹; and

Z¹² is selected from the group of H, halogen, alkyl, substituted alkyl, aryl, and substituted aryl;

with the proviso that when R¹ is

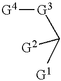

G¹, G², G³ and G⁴ cannot simultaneously have the following meanings:

G¹ and G²=H, G³=O and G⁴=H or Z²C═O where Z²=alkyl group.

A preferred compound of Formula III of the invention is Formula IIIa:

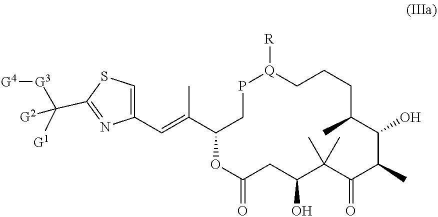

wherein the symbols have the following meaning:

P-Q is a C,C double bond or an epoxide;

R is a H atom or a methyl group;

G¹ is an H atom, an alkyl group, a substituted alkyl group or a halogen atom;

G² is an H atom, an alkyl group or a substituted alkyl group;

G³ is an O atom, an S atom or an NZ¹ group with

Z¹ being an H atom, an alkyl group, a substituted alkyl group, an acyl group, or a substituted acyl group, and

G⁴ is an H atom, an alkyl group, a substituted alkyl group, an OZ² group, an NZ²Z³ group, a Z²C═O group, a Z⁴SO₂ group or an optionally substituted glycosyl group with Z² being a H atom, an alkyl group, a substituted alkyl group, an aryl group, a substituted aryl group or a heterocyclic group;

Z³ an H atom, an alkyl group, a substituted alkyl group, an acyl group or a substituted acyl group; and

Z⁴ an alkyl, a substituted alkyl, an aryl, a substituted aryl or a heterocyclic group, with the proviso that G¹, G², G³ and G⁴ cannot have simultaneously the following meanings: G¹ and G²=H atom, G³=O atom and G⁴=H atom or Z²C═O with Z²=alkyl group.

A particularly preferred compound of Formula III is [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4,17-dioxabicyclo[14.1.0]heptadecane-5,9-dione (Compound 4) and pharmaceutically acceptable salts thereof.

When describing the compounds of the present invention, the phrase “lower alkyl” or “lower alk” (as part of another group) refers to an unsubstituted alkyl group of 1 to 6, preferably 1 to 4, carbon atoms.

The term “aralkyl” refers to an aryl group bonded directly through a lower alkyl group. A preferred aralkyl group is benzyl.

The term “aryl” refers to a monocyclic or bicyclic aromatic hydrocarbon group having 6 to 12 carbon atoms in the ring portion. Exemplary of aryl herein are phenyl, naphthyl and biphenyl groups.

The term “heterocyclo” refers to a fully saturated or unsaturated, aromatic or nonaromatic cyclic group which is a 4 to 7 membered monocyclic, 7 to 11 membered bicyclic, or 10 to 15 membered tricyclic ring system which has at least one heteroatom in at least one carbon atom-containing ring. Each ring of the heterocyclic group containing a heteroatom may have 1, 2, 3 or 4 heteroatoms selected from nitrogen, oxygen and sulfur where the nitrogen and sulfur heteroatoms may also optionally be oxidized and the nitrogen heteroatoms may also optionally be quaternized. The heterocyclo group may be attached at any heteroatom or carbon atom.

Exemplary monocyclic heterocyclo groups include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydrothiopyranyl, tetrahydropyranyl, morpholinyl, thiamorpholinyl, thiamorpholinyl sulfoxide, tetrahydrothiopyranylsulfone, thiamorpholinyl sulfone, 1,3-dioxlane, tetrahydro-1,1-dioxothienyl, dioxanyl, isothiazolidinyl, thietanyl, thiiranyl, triazinyl, triazolyl, and the like.

Exemplary bicyclic heterocyclo groups include benzothiazolyl, benzoxazolyl, benzothienyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

When a group is referred to as being optionally substituted, it may be substituted with one to five, preferably one to three, substituents such as F, Cl, Br, I, trifluoromethyl, trifluoromethoxy, hydroxy, lower alkoxy, cycloalkoxy, heterocyclooxy, oxo, lower alkanoyl, aryloxy, lower alkanoyloxy, amino, lower alkylamino, arylamino, aralkylamino, cycloalkylamino, heterocycloamino, disubstituted amines in which the two amino substituents independently are selected from lower alkyl, aryl or aralkyl, lower alkanoylamino, aroylamino, aralkanoylamino, substituted lower alkanoylamino, substituted arylamino, substituted aralkylanoylamino, thiol, lower alkylthio, arylthio, aralkylthio, cycloalkylthio, heterocyclothio, lower alkylthiono, arylthiono, aralkylthiono, lower alkylsulfonyl, arylsulfonyl, aralkylsulfonyl, sulfonamide (e.g., SO₂NH₂), substituted sulfonamide, nitro, cyano, carboxy, carbamyl (e.g., CONH₂), substituted carbamyl (e.g., CONH-lower alkyl, CONH-aryl, CONH-aralkyl or cases where there are two substituents on the nitrogen independently selected from lower alkyl, aryl or aralkyl), lower alkoxycarbonyl, aryl, substituted aryl, guanidine, and heterocyclos (e.g., indolyl, imidazolyl, furyl, thienyl, thiazolyl, pyrrolidyl, pyridyl, pyrimidyl and the like). Where noted above that the substituent is further substituted, it will be substituted with F, Cl, Br, I, optionally substituted lower alkyl, hydroxy, optionally substituted lower alkoxy, optionally substituted aryl, or optionally substituted aralkyl.

All stereoisomers of the Formula I, II, III and IV compounds of the instant invention are contemplated, either in admixture or in pure or substantially pure form. The definition of the formula I compounds embraces all possible stereoisomers and their mixtures. The Formula I, II, III and IV definitions very particularly embrace the racemic forms and the isolated optical isomers having the specified activity.

A particularly preferred epothilone analog for use in the methods of the invention is Compound 1: [1S-[1R*,3R*(E),7R*,10S*,11R*,12R*,16S*]]-7,11-dihydroxy-8,8,10,12,16-pentamethyl-3-[1-methyl-2-(2-methyl-4-thiazolyl)pethenyl]-4-aza-17-oxabicyclo[14.1.0]heptadecane-5,9-dione. Another exemplary epothilone is [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4,17-dioxabicyclo[14.1.0]heptadecane-5,9-dione, Compound 4.

Compound 1, an exemplary epothilone analog of the invention, is a semi-synthetic epothilone analog and has a mode of action analogous to paclitaxel (i.e., microtubule stabilization). However, in preclinical pharmacology studies, Compound 1 has demonstrated significant improvement over paclitaxel in several critical aspects. Compound 1 exhibits a very impressive and broad spectrum of antitumor activity against paclitaxel-sensitive (A2780, HCT116 and LS174T) and, more importantly, as well as paclitaxel-resistant human colon tumors (HCT116/VM46), ovarian carcinoma (Pat-7 and A2780Tax) and breast carcinoma (Pat-21) models. Compound 1 is orally efficacious; the antitumor activity produced after oral administration is comparable to that produced by parenteral administration of the drug. These preclinical efficacy data indicate that Compound 1 demonstrates improved clinical efficacy in TAXOL®-insensitive and sensitive disease types.

Compound 2: (R)-2,3-tetrahydro-1-(1H-imidazol-4-ylmethyl)-3-(phenylmethyl)-4-(2-thienylsulfonyl)-1H-1,4-benzodiazepine-7-carbonitrile, hydrochloride salt.

Compound 3: A CDK inhibitor is shown below

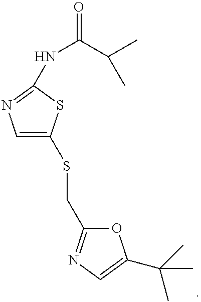

Compound 4: 1S-[1R*,3R*(E),7R*,10S*,11R*,12R*,16S*]]-3-[2-[2-(Aminomethyl)-4-thiazolyl]-1-methylethenyl]-7,11-dihydroxy-8,8,10,12,16-pentamethyl-4,17-dioxabicyclo[14.1.0]heptadecane-5,9-dione

Compound 5: N-[5-[[[5-(1,1-Dimethylethyl)-2-oxazolyl]methyl]thio]-2-thiazolyl]-4-piperidinecarboxamide.

Combinations of a microtubulin-stabilizing agent with another agent is contemplated by the present invention, and may include the addition of an anti-proliferative cytotoxic agent. Classes of compounds that may be used as anti-proliferative cytotoxic agents include the following:

co-stimulatory modulating agents including, without limitation, CTLA4 antagonists, ipilimumab, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70;

alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide;

antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gerncitabine; and

natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as TAXOL®), Mithramycin, Deoxyco-formycin, Mitomycin-C, L-Asparaginase, Interferons (especially IFN-a), Etoposide, and Teniposide.

Other anti-proliferative cytotoxic agents contemplated by the present invention are navelbene, CPT-11, anastrazole, letrazole, capecitabine, reloxafine, cyclophosphamide, ifosamide, and droloxafine.

Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

As is known in the art, Ipilimumab refers to an anti-CTLA-4 antibody, and is a fully human IgG_{1PG} antibody derived from transgenic mice having human genes encoding heavy and light chains to generate a functional human repertoire. Ipilimumab can also be referred to by its CAS Registry No. 477202-00-9, and is disclosed as antibody 10DI in PCT Publication No. WO01/14424, incorporated herein by reference in its entirety and for all purposes. Specifically, Ipilimumab describes a human monoclonal antibody or antigen-binding portion thereof that specifically binds to CTLA4, comprising a light chain variable region and a heavy chain variable region having a light chain variable region comprised of SEQ ID NO:5, and comprising a heavy chain region comprised of SEQ ID NO:6. Pharmaceutical compositions of Ipilimumab include all pharmaceutically acceptable compositions comprising Ipilimumab and one or more diluents, vehicles and/or excipients. Examples of a pharmaceutical composition comprising Ipilimumab are provided in PCT Publication No. WO2007/67959. Impilimumab may be administered by I.V.

Light Chain Variable Region for Impilimumab:
(SEQ ID NO: 1)
EIVLTQSPGTLSLSPGERATLSCRASQSVGSSYLAWYQQKPGQAPRLLI
YGAFSRATGIPDRFSGSGSGTDFTLTISRLEPEDFAVYYCQQYGSSPWT
FGQGTKVEIK
Heavy Chain Variable Region for Impilimumab:
(SEQ ID NO: 2)
QVQLVESGGGVVQPGRSLRLSCAASGFTFSSYTMHWVRQAPGKGLEWVT
FISYDGNNKYYADSVKGRFTISRDNSKNTLYLQMNSLRAEDTAIYYCAR
TGWLGPFDYWGQGTLVTVSS

As noted elsewhere herein, the administration of one or more anti-CTLA4 antagonists may be administered either alone or in combination with a peptide antigen (e.g., gp100), in addition to an anti-proliferative agent disclosed herein. A non-limiting example of a peptide antigen would be a gp100 peptide comprising, or alternatively consisting of, the sequence selected from the group consisting of: IMDQVPFSV (SEQ ID NO:3), and YLEPGPVTV (SEQ ID NO:4). Such a peptide may be administered orally, or preferably by injection s.c. at 1 mg emulsified in incomplete Freund's adjuvant (IFA) injected s.c. in one extremity, and 1 mg of either the same or a different peptide emulsified in WA may be injected in another extremity.

The present invention also encompasses a pharmaceutical composition useful in the treatment of cancer, comprising the administration of a therapeutically effective amount of a microtubulin-stabilizing agent, either alone or in combination with another agent, with or without pharmaceutically acceptable carriers or diluents. The compositions of the present invention may further comprise one or more pharmaceutically acceptable additional ingredient(s) such as alum, stabilizers, antimicrobial agents, buffers, coloring agents, flavoring agents, adjuvants, and the like. The Formula I, II, III, and/or IIIa, or analogs thereof compounds, CTLA4 antagonist agents, antineoplastic agents, and compositions of the present invention may be administered orally or parenterally including the intravenous, intramuscular, intraperitoneal, subcutaneous, rectal and topical routes of administration.

For oral use, the antineoplastic agents, Formulas I, II, III, and/or IIIa or analogs thereof compounds and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The compounds of Formulas I, II, III, and/or IIIa or analogs thereof, as well as the anti-CTLA4 agents and anti-neoplastic agents, described herein may also be delivered transdermally. The transdermal compositions can take the form of creams, lotions, aerosols and/or emulsions and can be included in a transdermal patch of the matrix or reservoir type as are conventional in the art for this purpose.

The combinations of the present invention may also be used in conjunction with other well known therapies that are selected for their particular usefulness against the condition that is being treated.

If formulated as a fixed dose, the active ingredient(s) of the microtubulin-stabilizing agents, or combination compositions, of this invention are employed within the dosage ranges described below. Alternatively, the anti-CTLA4 agent, and Formulas I, II, III, and/or IIIa or analogs thereof compounds may be administered separately in the dosage ranges described below. In a preferred embodiment of the present invention, the anti-CTLA4 agent is administered in the dosage range described below following or simultaneously with administration of the Formulas I, II, III, and/or IIIa or analogs thereof compound in the dosage range described below.

The following sets forth preferred therapeutic combinations and exemplary dosages for use in the methods of the present invention. Where “Compound of Formula II” appears, any of the variations of Formula II or Formula III set forth herein are contemplated for use in the chemotherapeutic combinations. Preferably, Compound 1 or Compound 4 is employed.

| | DOSAGE | |
|---|---|---|
| THERAPEUTIC COMBINATION | mg/m² (per dose) | |
| Compound of Formula I (Ixabepilone) | 1-500 | mg/m² |
| Compound of Formula II) | 0.1-100 | mg/m² |
| Compound of Formula III | 0.1-100 | mg/m² |
| Compound of Formula IIIa | 0.1-100 | mg/m³ |
| Compound of Formula I (Ixabepilone) + | 1-500 | mg/m² |
| anti-CTLA4 Antibody | 0.1-25 | mg/kg |
| Compound of Formula II + | 0.1-100 | mg/m² |
| anti-CTLA4 Antibody | 0.1-25 | mg/kg |
| Compound of Formula III + | 0.1-100 | mg/m² |
| anti-CTLA4 Antibody | 0.1-25 | mg/kg |
| Compound of Formula IIIa (Paclitaxel) + | 0.1-100 | mg/m² |
| anti-CTLA4 Antibody | 0.1-25 | mg/kg |

While this table provides exemplary dosage ranges of the Formula I, Formula II, Formula III and Formula IIIa compounds and certain anticancer agents of the invention, when formulating the pharmaceutical compositions of the invention the clinician may utilize preferred dosages as warranted by the condition of the patient being treated. For example, the compound of Formula I may preferably be administered at about 40 mg/m² every 3 weeks. Compound 1 may preferably be administered at about 25-60 mg/m² every 3 weeks. Compound 2, may preferably be administered at a dosage ranging from about 25-500 mg/m² every three weeks for as long as treatment is required.

The anti-CTLA4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 9 mg/kg.

In another specific embodiment, the escalating dosage regimen includes administering a first dosage of CTLA-4 antibody at about 5 mg/kg and a second dosage of CTLA-4 antibody at about 9 mg/kg.

Further, the present invention provides an escalating dosage regimen, which includes administering an increasing dosage of CTLA-4 antibody about every six weeks.

In an aspect of the present invention, a stepwise escalating dosage regimen is provided, which includes administering a first CTLA-4 antibody dosage of about 3 mg/kg, a second CTLA-4 antibody dosage of about 3 mg/kg, a third CTLA-4 antibody dosage of about 5 mg/kg, a fourth CTLA-4 antibody dosage of about 5 mg/kg, and a fifth CTLA-4 antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Certain cancers can be treated effectively with compounds of Formulas I, II, III, and/or IIIa and a one or more anti-CTLA4 agents. Such triple and quadruple combinations can provide greater efficacy. When used in such triple and quadruple combinations the dosages set forth above can be utilized.

When employing the methods or compositions of the present invention, other agents used in the modulation of tumor growth or metastasis in a clinical setting, such as antiemetics, can also be administered as desired.

The present invention encompasses a method for the synergistic treatment of cancer wherein anti-CTLA4 agent and a Formulas I, II, III, and/or IIIa compound are administered simultaneously or sequentially. Thus, while a pharmaceutical formulation comprising anti-CTLA4 agent(s) and a Formulas I, II, III, and/or IIIa compound may be advantageous for administering the combination for one particular treatment, prior administration of the anti-CTLA4 agent(s) may be advantageous in another treatment. It is also understood that the instant combination of anti-CTLA4 agent(s) and Formulas I, II, III, and/or Ma compound may be used in conjunction with other methods of treating cancer (preferably cancerous tumors) including, but not limited to, radiation therapy and surgery. It is further understood that a cytostatic or quiescent agent, if any, may be administered sequentially or simultaneously with any or all of the other synergistic therapies.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-CTLA4 agent(s)) on the patient, and in view of the observed responses of the disease to the administered therapeutic agents.

In the methods of this invention, a compound of Formula I, II, III or Formula IIIa is administered simultaneously or sequentially with an anti-CTLA4 agent. Thus, it is not necessary that the anti-CTLA4 therapeutic agent(s) and compound of Formulas I, II, III, and/or IIIa, be administered simultaneously or essentially simultaneously. The advantage of a simultaneous or essentially simultaneous administration is well within the determination of the skilled clinician.

Also, in general, the compound of Formulas I, II, III, and/or IIIa, and anti-CTLA4 agent(s) do not have to be administered in the same pharmaceutical composition, and may, because of different physical and chemical characteristics, have to be administered by different routes. For example, the compound of Formula I, II, III, or IV may be administered intravenously to generate and maintain good blood levels thereof, while the anti-CTLA4 agent(s) may also be administered intravenously. Alternatively, the compound of Formula I, II, III, or IV may be administered orally to generate and maintain good blood levels thereof, while the anti-CTLA4 agent(s) may also be administered intravenously. Alternatively, the compound of Formula I, II, III, or IV may be administered intravenously to generate and maintain good blood levels thereof, while the anti-CTLA4 agent(s) may also be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compound of Formulas I, II, III, and/or IIIa or analogs thereof and anti-CTLA4 agent(s) will depend upon the diagnosis of the attending physicians and their judgment of the condition of the patient and the appropriate treatment protocol.

If the compound of Formula I, II, III, and/or IIIa and the anti-CTLA4 agent(s) are not administered simultaneously or essentially simultaneously, then the initial order of administration of the compound of Formulas I, II, III, and/or IIIa, and the anti-CTLA4 agent(s) may be varied. Thus, for example, the compound of Formulas I, II, III, and/or IIIa or analogs thereof may be administered first followed by the administration of the anti-CTLA4 agent(s); or the anti-CTLA4 agent(s) may be administered first followed by the administration of the compound of Formulas I, II, III, and/or IIIa. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the anti-CTLA4 agent(s) may be administered initially. The treatment is then continued with the administration of the compound of Formulas I, II, III, and/or IIIa or analogs thereof and optionally followed by administration of a cytostatic agent, if desired, until the treatment protocol is complete. Alternatively, the administration of the compound of Formulas I, II, III, and/or IIIa or analogs thereof and optionally followed by administration of a cytostatic agent may be administered initially. The treatment is then continued with the administration of the anti-CTLA4 agent(s), until the treatment protocol is complete.

Thus, in accordance with experience and knowledge, the practicing physician can modify each protocol for the administration of a component (therapeutic agent—i.e., compound of Formulas I, II, III, and/or IIIa or analogs thereof, anti-CTLA4 agent(s)) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Thus, the present invention provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

Most preferably, the invention is used to treat accelerated or metastatic cancers of the breast and/or lung.

### Biomarkers and Biomarker Sets

The invention includes individual biomarkers and biomarker sets having both diagnostic and prognostic value in proliferative disease areas in which microtubulin status is of importance, e.g., in cancers or tumors, or in disease states in which cell signaling and/or cellular proliferation controls are abnormal or aberrant. The biomarker sets comprise a plurality of biomarkers that highly correlate with resistance or sensitivity to one or more microtubulin-stabilizing agents.

The biomarkers and biomarker sets of the invention enable one to predict or reasonably foretell the likely effect of one or more microtubulin-stabilizing agents in different biological systems or for cellular responses merely based upon whether one or more of the biomarkers of the present invention are overexpressed relative to normal. The biomarkers and biomarker sets can be used in in vitro assays of cellular proliferation by sample cells to predict in vivo outcome. In accordance with the invention, the various biomarkers and biomarker sets described herein, or the combination of these biomarker sets with other biomarkers or markers, can be used, for example, to predict and monitor how patients with cancer might respond to therapeutic intervention with one or more microtubulin-stabilizing agents.

In specific embodiments of the present invention, overexpression of TUBB3, BCRP (also referred to as ABCG2), MDR1 (also referred to as ABC1), MRP1 (also referred to as ABCC1), and/or β-tubulin mutations correlated with response to microtubulin-stabilizing agents. Specifically, overexpression of TUBB3 resulted in xenografts that were resistant to docetaxel and vinorelbine, yielding antitumor efficacy ranging 0.2-0.9 and 0.1-0.3 LCK, respectively. In contrast, ixabepilone was active in all 4 tumors in which TUBB3 was overexpressed, yielding 1.6-4.2 LCK (see Table 1) when tested at their maximum tolerated doses (MTD). The BCRP overexpressing HEK293/BCRP cell line demonstrated resistance to paclitaxel and mitoxantrone by 9.8-fold (1050=8.7 nM) and 4.1-fold (IC50-26.4 nM), respectively, in comparison with the vector-transfected control line. This resistance could be reversed by fumitremorgin C, a selective inhibitor of BCRP. In contrast, ixabepilone was far less susceptible to the BCRP-mediated resistance, resulting in a resistance factor of only 1.9 fold (1050=4.1 nM). Together, these results suggest ixabepilone may offer breast and/or lung cancer patients a potentially valuable treatment option.

Measuring the level of expression of a biomarker and biomarker set provides a useful tool for screening one or more tumor samples before treatment of a patient with the microtubulin-stabilizing agents. The screening allows a prediction of whether the cells of a tumor sample will respond favorably to the mierotubulin-stabilizing agents, based on the presence or absence of over-expression—such a prediction provides a reasoned assessment as to whether or not the tumor, and hence a patient harboring the tumor, will or will not respond to treatment with the microtubulin-stabilizing agents.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

The biomarker or biomarker set can also be used as described herein for monitoring the progress of disease treatment or therapy in those patients undergoing treatment for a disease involving a microtubulin-stabilizing agent.

The biomarkers also serve as targets for the development of therapies for disease treatment. Such targets may be particularly applicable to treatment of cancer, such as, for example, breast and/or lung cancer.

Indeed, because these biomarkers are differentially expressed in sensitive and resistant cells, their expression patterns are correlated with relative intrinsic sensitivity of cells to treatment with microtubulin-stabilizing agents. Accordingly, the biomarkers over expressed in resistant cells may serve as targets for the development of new therapies for the tumors which are resistant to microtubulin-stabilizing agents. The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

The present invention encompasses the use of any one or more of the following as a biomarker for use in predicting microtubulin-stabilizing agent response: TUBB3, BRCP, MDR1, MRP1, and beta-tubulin mutations.

The present invention also encompasses any combination of the aforementioned biomarkers, including, but not limited to: TUBB3, BRCP, MDR1, MRP1, and beta-tubulin mutations; TUBB3, BRCP, MDR1, MRP1; TUBB3, BRCP, MDR1; BRCP, MDRI, MRP1, and beta-tubulin mutations; BRCP, MDR1, MRP1; MDR1, MRP1, and beta-tubulin mutations; TUBB3 and BRCP; TUBB3 and MDR1; TUBB3 and MRP1; TUBB3 and beta-tubulin mutations; BRCP and MDR1; BRCP and MRP1; BRCP and beta-tubulin mutations; MDR1 and MRP1; MDR1 and beta-tubulin mutations; and/or MRP1 and beta-tubulin mutations.

Identification of biomarkers that provide rapid and accessible readouts of efficacy, drug exposure, or clinical response is increasingly important in the clinical development of drug candidates. Embodiments of the invention include measuring changes in the levels of mRNA and/or protein in a sample to determine whether said sample contains increased expression of TUBB3, BRCP, MDR1, MPR1, and/or beta-tubulin mutations. In one aspect, said samples serve as surrogate tissue for biomarker analysis. These biomarkers can be employed for predicting and monitoring response to one or more microtubulin-stabilizing agents. In one aspect, the biomarkers of the invention are one or more of the following: TUBB3, BRCP, MDR1, MPR1, and/or beta-tubulin mutations, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to microtubulin-stabilizing agents.

Methods of measuring the level of any given marker described herein may be performed using methods well known in the art, which include, but are not limited to PCR; RT-PCR; FISH; IHC; immuno-detection methods; immunoprecipitation; Western Blots; ELISA; radioimmunoassays; PET imaging; HPLC; surface plasmon resonance, and optical spectroscopy; and mass spectrometry, among others.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, John Wiley & Sons, Inc., New York at 10.16.1 (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, John Wiley & Sons, Inc., New York at 10.8.1 (1994).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, John Wiley & Sons, Inc., New York at 11.2.1 (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, 2-D Proteome Analysis Protocols, A. J. Link, ed., Humana Press (1999), ISBN: 0896035247; Mass Spectrometry of Proteins and Peptides, J. R. Chapman, ed., Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

In order to facilitate a further understanding of the invention, the following examples are presented primarily for the purpose of illustrating more specific details thereof. The scope of the invention should not be deemed limited by the examples, but to encompass the entire subject matter defined by the claims.

### EXAMPLE 1

### Method of Assessing the Response of TUBB3 Overexpressing Tumor Xenografts to the Administration of Microtubulin Stabilizing Agents In Vivo

### Methods

Cancer cell lines overexpressing TUBB3 were evaluated in vivo in mice for sensitivity to ixabepilone, docetaxel and vinorelbine. These include DU4475 and PAT21 breast, as well as H1155 and LX-1 lung cancer lines. BCRP overexpressing HEK-293 cell line was studied in vitro for sensitivity to ixabepilone, paclitaxel and mitoxantrone.

Compounds and reagents. Ixabepilone, docetaxel and mitoxantrone were solubilized in 100% DMSO at 10 mg/ml for in vitro studies.

Cell culture: HEK and HEK/BCRP cells were maintained in RPMI-1640 (Gibco) supplemented with 10% heat-inactivated fetal bovine serum and 25 mM HEPES at 37° C./5% CO2.

Cell growth assays. Cell growth assays were conducted in 6-well culture plates. Cells were plated at a density of 4×104 cells/well overnight. Compounds were then added (total DMSO content not exceeding 0.1%). Cell growth was determined by the direct counting of cell number, following trypsinization, using a Coulter Channelyzer.

Determination of BCRP Expression by Flow Cytometry.

Cells were acquired using CellQuest Pro on a FACSCalibur (BD) and analyzed using FlowJo software.

Immunohistochemistry Methods. Tumors were fixed overnight in 10% neutral buffered formalin and processed for paraffin embedding. Specimens were sectioned at 5 microns. Immunohistochemistry was performed on sections utilizing the Dako Envision Mouse Polymer kit with an antibody against Class III Beta-Tubulin, clone TUJ1 (Covance #MMS-435P), and counterstained with Gill 2 Hematoxylin. Images were taken at 200× magnification using ImagePro Plus software on an Olympus BX-60 microscope.

### Study Design

In vivo efficacy of ixabepilone was evaluated in TUBB3 overexpressing human breast (DU4475 and PAT21) and lung (H1155 and LX-1) tumor xenograft models
Tumor response was determined by measurement of tumors by caliper twice a week
Tumor weights were estimated using the formula

Tumor weight=(length×width2)/2
Tumor response endpoint was expressed in terms of log cell kill (LCK) expressed as

LCK=(T−C)÷(3.32×TVDT)
T−C, the difference in the time (days) required for the treated tumors (T) to reach a predetermined target size compared with those of the control group (C)

Tumor volume doubling time=TVDT: Median time (days) for control tumors to reach target size−Median time (days) for control tumors to reach half the target size
Activity was defined as achievement of LCK≧1
MTD—the dose level immediately above which excessive toxicity (i.e., more then one death) occurred
Statistical evaluations were performed using Gehan's generalized Wilcoxon test

### Results

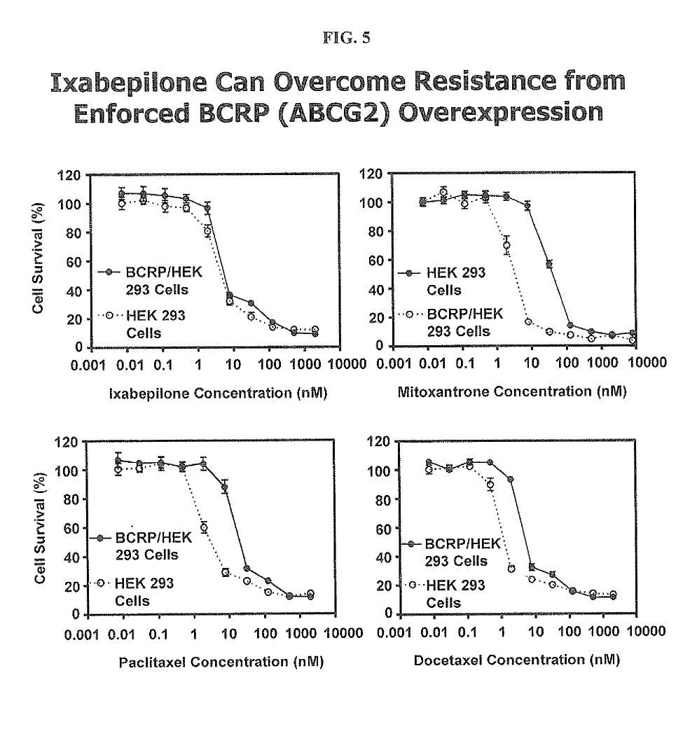

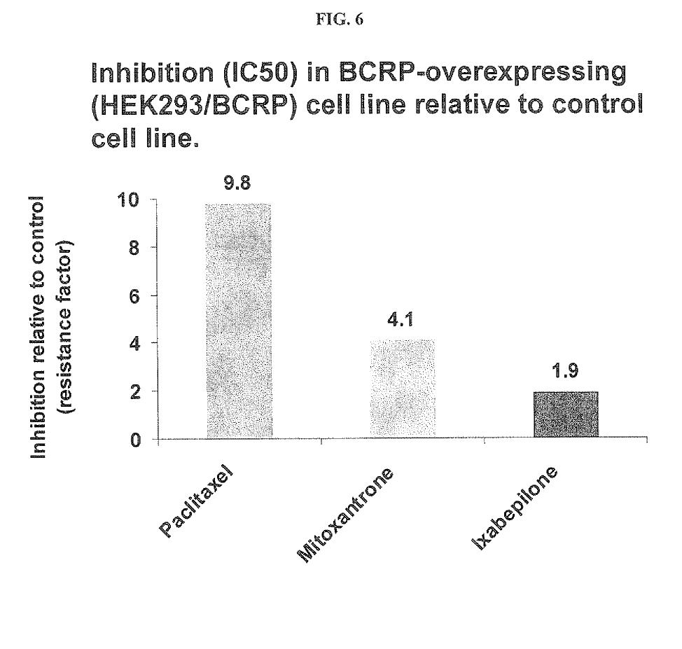

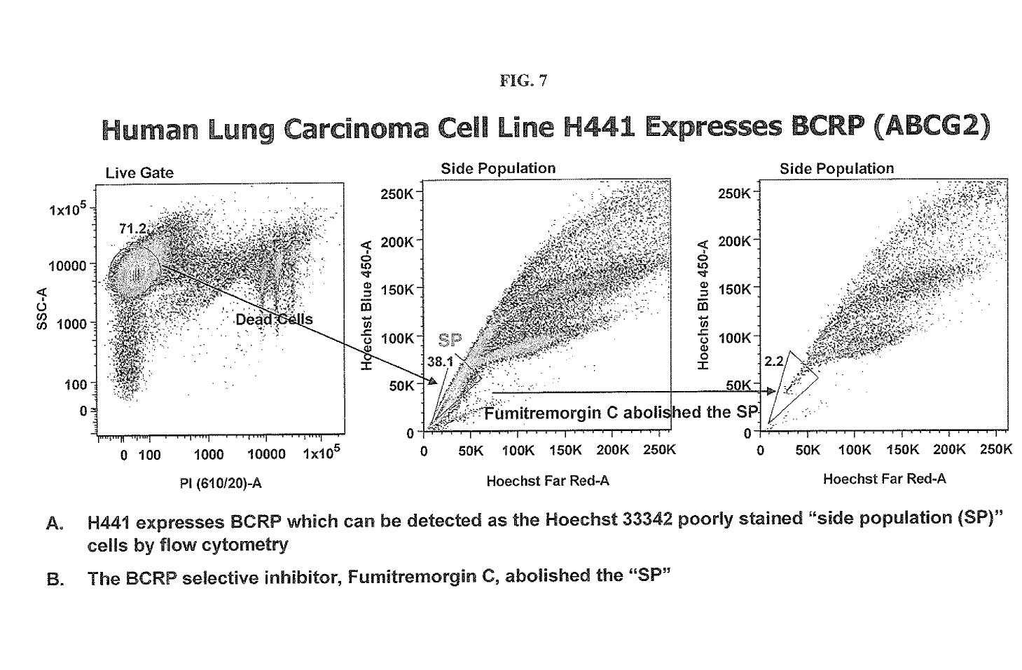

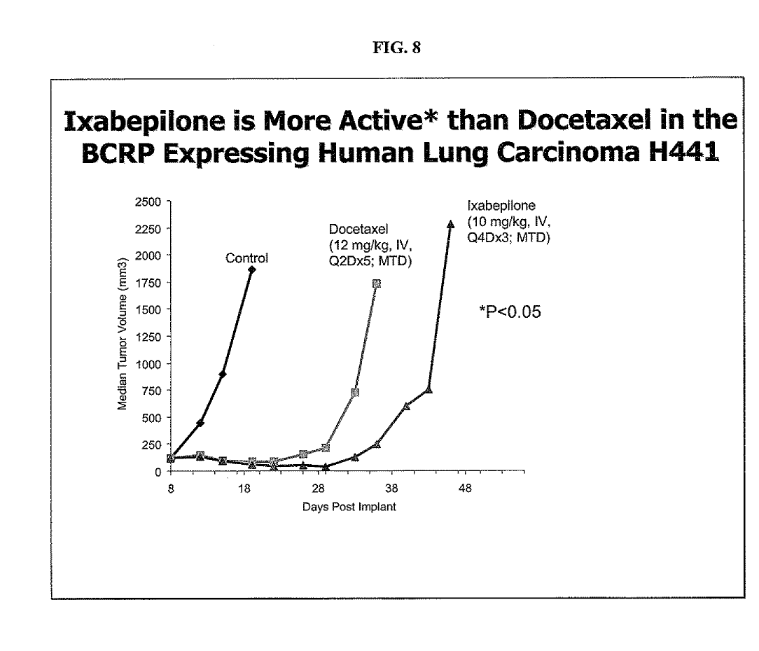

Efficacy evaluation in nude mice demonstrated that the 4 xenografts overexpressing TUBB3 were resistant to docetaxel and vinorelbine, yielding antitumor efficacy ranging 0.2-0.9 and 0.1-0.3 LCK, respectively (see FIG. 5). In contrast, ixabepilone was active in all 4 tumors, yielding L6-4.2 LCK (Table 1) when tested at their maximum tolerated doses (MTD). The BCRP overexpressing HEK293/BCRP cell line demonstrated resistance to paclitaxel and mitoxantrone by 9.8-fold (IC50=8.7 nM) and 4.1-fold (IC50=26.4 nM), respectively, in comparison with the vector-transfected control line (see FIG. 6). This resistance can be reversed by fumitremorgin C, a selective inhibitor of BCRP (see FIG. 7). In contrast, ixabepilone was far less susceptible to the BCRP-mediated resistance, resulting in a resistance factor of only 1.9 fold (IC50=4.1 nM) (see FIG. 8).

### Conclusion

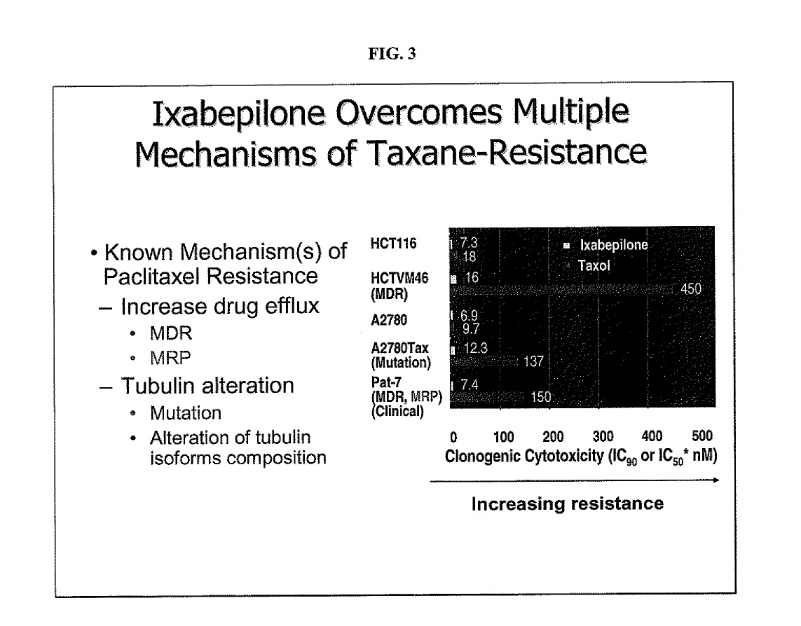

Compared with agents commonly used in breast cancer—paclitaxel, docetaxel, mitoxantrone, and vinorelbine—ixabepilone has markedly lower susceptibility to multiple resistance mechanisms that affect these agents (see FIG. 3). These include overexpression of TUBB3, BCRP (also referred to as ABCG2), MDR1 (also referred to as ABCB1), and MRP1 (also referred to as ABCC1), and β-tubulin mutations.

In vitro and in vivo models demonstrated that ixabepilone is minimally affected by overexpression of TUBB3, BCRP, MDR1 and MRP1, and β-tubulin mutations.

Ixabepilone's clinical activity has been demonstrated in metastatic breast cancer patients who developed resistance to other chemotherapy regimens, including anthracyclines and taxanes.

As a result of this activity, ixabepilone was recently FDA-approved in the United States combination with capecitabine for the treatment of patients with metastatic or locally advanced breast cancer after failure of an anthracycline and a taxane, and as monotherapy for the treatment of metastatic or locally advanced breast cancer in patients after failure of an anthracycline, a taxane, and capecitabine.

The results from the present study add to accumulating data supporting the inclusion of ixabepilone as a key component of breast cancer treatment. Moreover, the results demonstrate the utility of diagnosing patients for the presence of BCRP-overexpression, in addition to MDR1, MRP1, other transporters, TUBB3 overexpression, and tubulin mutations, who may benefit from the administration of ixabepilone in the efficacious treatment of cancer, including breast and lung cancer.

TABLE 1
Comparison of the Antitumor Efficacy of Ixabepilone,
Docetaxel and Vinorelbine in 4 Human Tumor
Xenografts Overexpressing TUBB3

| | Ixabepilone | Docetaxel | Vinorelbine |
|---|---|---|---|
| | Antitumor | Antitumor | Antitumor |
| Tumor | Efficacy (LCK) | Efficacy (LCK) | Efficacy (LCK) |
| H1155 | 4.2 | 0.2 | 0.1 |
| DU4475 | 2.6 | 0.9 | 0.2 |
| Pat-21 | 1.6 | 0.3 | 0.3 |
| LX-1 | 2.6 | 0.5 | 0.1 |

### EXAMPLE 2

### Method to assess Multiple Resistance Expression Profile Using MRNA from Tissue and Cell Sources

Total RNA may be purified using RNEASY® system (Qiagen, Calif., USA). Mixed Oligo-d(T)₁₅ primers may be used to generate single-stranded cDNAs using the SUPERSCRIPT® First-strand Synthesis kit (Invitrogen, Calif., USA). Levels for each gene of interest and GAPDH transcripts may be analyzed using an Applied Biosystems 7900HT Sequence Detection System. Mixed primer/probe sets for each transcript of interest (TUBB3, catalog #Hs00964962_g1; BCRP, catalog #Hs00184979_m1; MDR-1, catalog 4 HS00184491_m1; MRP-1, catalog #Hs00219905_m 1; GAPDH, catalog #4326317E) may be obtained from Applied Biosystems and used according to the manufacturer's instructions.

Expression levels of transcripts of interest may then be normalized to endogenous GAPDH transcripts. Comparisons may be made between samples by ΔΔCt comparative analysis using manufacturer's software (Applied Biosystems). Briefly, ΔCT=(MDR CT)−(GAPDH CT); ΔΔCT=(ΔCT^{Probel}−ΔCT^{Probe2}); and Fold change=2^{ΔΔC}.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

What is claimed is:

1. A method for predicting the likelihood a patient will respond therapeutically to a cancer treatment comprising the administration of a microtubulin-stabilizing agent, wherein said prediction method comprises the steps of:
(a) measuring the level of a biomarker in a sample from said patient; and
(b) comparing the level of said biomarker in said sample relative to a standard to permit assignment of said sample to either being a member of an overexpression positive class or an overexpression negative class,
wherein an overexpression positive sample member indicates an increased likelihood said patient will respond therapeutically to said cancer treatment, wherein said cancer treatment is the administration of ixabepilone.

2. The method according to claim 1, wherein said cancer is breast or lung cancer.

3. The method according to claim 1, wherein said biomarker is selected from the group consisting of: TUBB3, BRCP, MDR1, MPR1, and beta-tubulin mutations.

4. The method according to claim 3, wherein said method further comprises measuring the expression level of an additional biomarker selected from the group consisting of TUBB3, BRCP, MDR1, MPR1, and beta-tubulin mutations.

5. A method for treating a patient with cancer comprising the steps of:
(a) measuring the level of a biomarker in a sample from said patient; and
(b) comparing the level of said biomarker in said sample relative to a standard to permit assignment of said sample to either being a member of an overexpression positive class or an overexpression negative class,
wherein an overexpression positive sample member indicates an increased likelihood said patient will respond therapeutically to said cancer treatment, wherein said cancer treatment is the administration of ixabepilone.

6. The method according to claim 5, wherein said cancer is breast or lung cancer.

7. The method according to claim 5, wherein said biomarker is selected from the group consisting of: TUBB3, BRCP, MDR1, MPR1, and beta-tubulin mutations.

8. The method according to claim 7, wherein said method further comprises measuring the expression level of an additional biomarker selected from the group consisting of: TUBB3, BRCP, MDR1, MPR1, and beta-tubulin mutations.

9. A kit for use in treating a patient with cancer, comprising:
(a) a means for determining whether a sample from said patient is positive for overexpression of a biomarker;
(b) a therapeutically effective amount of a ixabepilone, or a pharmaceutically acceptable salt, hydrate, or solvate thereof, or a pharmaceutically acceptable salt or hydrate or solvate thereof; and
(c) instructions for use of said kit.

10. The kit according to claim 9, wherein said cancer is breast or lung cancer.

11. The kit according to claim 9, wherein said biomarker is selected from the group consisting of: TUBB3, BRCP, MDR1, MPR1, and beta-tubulin mutations.

12. The method according to claim 11, wherein said method further comprises measuring the expression level of an additional biomarker selected from the group consisting of TUBB3, BRCP, MDR1, MPR1, and beta-tubulin mutations.

13. The method according to claim 1, 5, or 9, wherein said measurement is performed using a method selected from the group consisting of: (a) PCR; (b) RT-PCR; (c) FISH; (d) IHC; (e) immuno-detection methods; (f) Western Blot; (g) ELISA; (h) radioimmuno assays; (i) immunoprecipitation; (j) PET imaging; (k) HPLC; (l) surface plasmon resonance; (m) optical spectroscopy; and (i) mass spectrometry.


SEQUENCE LISTING (4 sequences)

SEQ ID NO: 1 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - protein, 9 aa, Homo sapiens
IMDQVPFSV

SEQ ID NO: 4 - protein, 9 aa, Homo sapiens
YLEPGPVTV